(19) United States
(12) Patent Application Publication
(10) Pub. No.: US 2006/0275520 A1
(43) Pub. Date: Dec. 7, 2006

(54) White peony extract for improving the duration and quality of sleep

(75) Inventors: Chioma Jane Ikonte, Corona, CA (US); Jatinder Rana, Grand Rapids, MI (US); David J. Fast, Grand Rapids, MI (US); Yingqin Li, Murrieta, CA (US)

Correspondence Address: IN RE: ALTICOR INC. 28533;BRINKS, HOFER, GILSON & LIONE, P.O. BOX 10395, CHICAGO, IL, 60610, US

(73) Assignee: Access Business Group International LLC

(21) Appl. No.: 11/437,885

(22) Filed: May 19, 2006

Related U.S. Application Data
(63) Continuation-in-part of application No. 11/075,010, filed on Mar. 7, 2005.

(51) Int. Cl. A61K 36/725; A61K 36/70; A61K 36/65

(52) U.S. Cl. 424/773; 424/774; 424/778; 424/451; 424/440; 424/464

(57) ABSTRACT

The present invention relates to compositions for treating insomnia or sleeplessness. The compositions of the present invention comprise extracts of white peony root and in particular comprise paeoniflorin, either alone or in combination with at least one of jujube extract, radix polygala extract, and passion flower extract. The compositions of the present invention bind to GABA receptors and work synergistically to shorten the time needed to fall asleep and to improve the quality of sleep by reducing awakenings during sleep and increasing the duration of sleep.

This application claims priority to and is a continuation-in-part of U.S. patent application Ser. No. 11/075,010, filed Mar. 7, 2005.

### BACKGROUND

Insomnia or sleeplessness is a common condition generally caused by over stimulation of the mind and/or body. Factors such as stress, poor dietary habits, lack of physical activity, and psychological influences are major causes of insomnia. Insomnia also commonly reflects a lack of central inhibitory processes. For example, gamma-amino butyric acid (GABA) is the main inhibitory neurotransmitter of the central nervous system (CNS). Several studies have shown that activation of GABA receptors, through binding of GABA, favors sleep. In addition, during times of stress and anxiety, the body produces less GABA, which can make it more difficult to calm down due to a lessening of the inhibitory effects of GABA exerted through binding of GABA receptors. The result often is insomnia. The inability to sleep is a significant problem because sleep is necessary for survival and good health.

How long a person sleeps and how rested a person feels on waking can be influenced by many factors, including excitement or emotional distress. Medications also can play a part; some medications make a person sleepy while others make sleeping difficult. Even some food elements or additives such as caffeine, strong spices, and monosodium glutamate (MSG) may affect sleep.

When sleep disorders interfere with a person's normal activities and sense of well-being, the intermittent use of sleep medications such as sedatives or hypnotics, may be useful. A sedative drug decreases activity, moderates excitement, and calms the recipient, whereas a hypnotic drug produces drowsiness and facilitates the onset and maintenance of a state of sleep that resembles natural sleep.

Nonbenozodiazepine is one example of a currently available sedative drug that depresses the CNS in a relative, nonselective, dose-dependent fashion. Nonbenozodiazepine produces progressively calming effects or feelings of drowsiness (sedation) until sleep is reached. However, individuals that take nonbenzodiazepine can build up a tolerance to the sedative effects of this drug. This can be dangerous because when taken in higher doses, nonbenozodiazepine can cause unconsciousness, coma, surgical anesthesia, or fatal depression of respiration and cardiovascular regulation.

Hypnotics, which include minor tranquilizers and anti-anxiety drugs, are among the most commonly used drugs for treating sleep disorders or achieving a good night's sleep. Most are quite safe, but all can lose their effectiveness once a person builds up a tolerance to them. Moreover, hypnotic drugs are associated with withdrawal symptoms when use is discontinued. Indeed, discontinuing use of a hypnotic drug after more than a few days' use can make the original sleep problem worse and can increase feelings of anxiety. Additionally, most hypnotics require a doctor's prescription because they may be habit-forming or addictive, and overdose is possible. Hypnotics are particularly risky for the elderly and for people with breathing problems because they tend to suppress brain areas that control breathing. They also reduce daytime alertness, making driving or operating machinery hazardous. Hypnotics are especially dangerous when taken with alcohol, other hypnotics, narcotics, antihistamines, and anti-depressants. All of these drugs cause drowsiness and can suppress breathing, making the combined effects more dangerous.

Because sleep is vital to a healthy lifestyle, compositions for and methods of treating insomnia or sleeplessness, other than use of sedative or hypnotic pharmaceuticals, are both important and useful.

### BRIEF SUMMARY

The present invention encompasses the use of extracts of white peony (Paeonia lactiflora), specifically a paeoniflorin (or peoniflorin) extract, in compositions and methods for treating or preventing insomnia or sleeplessness.

In particular, the present invention involves the use of paeoniflorin, a chemical marker used in numerous white peony root extracts, as an active ingredient for treating or preventing insomnia. Therefore, in one example, the present invention is a composition comprising paeoniflorin, wherein the composition specifically binds to GABA receptors.

In another example, a method of the present invention comprises administering an extract of white peony (Paeonia lactiflora) (e.g., paeoniflorin) in combination with extracts of one or more of the following: wild jujube, radix polygala, and passion flower. In combination, these extracts work synergistically to shorten the time needed to fall asleep and improve the quality of sleep by reducing the number of awakenings during sleep and increasing the duration of sleep. Thus, the present invention affords a safe and natural way of improving sleep quality without the use of hormones or pharmaceutical sedatives or hypnotics.

Accordingly, in one example, the present invention provides a composition comprising a paeoniflorin extract, an acceptable carrier and one or more of the following: jujube extract or any of its derivatives, radix polygala extract or any of its derivatives, or passion flower extract or any of its derivatives, wherein the composition is effective for treating insomnia or sleeplessness.

In another example, the present invention is a composition comprising a paeoniflorin extract, an acceptable carrier, and one or more of the following: jujube extract or any of its derivatives, or passion flower extract or any of its derivatives, wherein the composition binds to GABA receptors.

In a further example, the present invention is a method of treating insomnia or sleeplessness comprising administering to a subject a composition comprising a paeoniflorin extract, an acceptable carrier and one or more of the following: jujube extract or any of its derivatives, radix polygala extract or any of its derivatives, and passion flower extract or any of its derivatives.

In another example, the present invention is a method of treating insomnia or sleeplessness comprising administering to a subject a composition that binds to GABA receptors, wherein the composition comprises a paeoniflorin extract, an acceptable carrier, and one or more of the following: jujube extract or any of its derivatives, or passion flower extract or any of its derivatives.

In yet a further example, the present invention is a method of maximizing the sleep inducing or sleep improving effects of white peony extracts comprising standardizing an extract of a white peony root to at least approximately 5% paeoniflorin. In other examples, the white peony root extract may be standardized to approximately 5-100% peaonliflorin, for example, 10%, 15%, 20%, 25%, 30%, 35%, 40%, 45%, 50%, 55%, 60%, 65%, 70%, 75%, 80%, 85%, 90%, 92%, 95%, 98%, or 100% paeoniflorin.

### BRIEF DESCRIPTION OF THE DRAWINGS

FIG. 1 is a graph illustrating the percent of specific binding between white peony 2 and GABA-A receptors at concentrations of 0.01, 0.03, 0.1, 0.3, 1, 3, and 10 μg/ml.

FIG. 2 is a graph illustrating the percent of specific binding between white peony 4% and GABA-A receptors at concentrations of 0.01, 0.03, 0.1, 0.3, 1, 3, and 10 μg/ml.

FIG. 3 is a graph illustrating the percent of specific binding between jujube fruit 6:1 and GABA-A receptors at concentrations of 0.01, 0.03, 0.1, 0.3, 1, 3, and 10 μg/ml.

FIG. 4 is a graph illustrating the percent of specific binding between wild jujube 2% extract and GABA-A receptors at concentrations of 0.01, 0.03, 0.1, 0.3, 1, 3, and 10 μg/ml.

FIG. 5 is a graph illustrating the percent of specific binding between radix polygala and GABA-A receptors at concentrations of 0.01, 0.03, 0.1, 0.3, 1, 3, and 10 μg/ml.

FIG. 6 is a graph illustrating the percent of specific binding between passion flower and GABA-A receptors at concentrations of 0.01, 0.03, 0.1, 0.3, 1, 3, and 10 μg/ml.

FIG. 7 is a graph illustrating the percent of specific binding between valerian extract and GABA-A receptors at concentrations of 0.01, 0.03, 0.1, 0.3, 1, 3, and 10 μg/mi.

FIG. 8 is a graph illustrating the percent of specific binding between Formula 1 (434 mg white peony and 200 mg passion flower) and GABA-A receptors at concentrations of 0.01, 0.03, 0.1, 0.3, 1, 3, and 10 μg/ml.

FIG. 9 is a graph illustrating the percent of specific binding between Formula 2 (375 mg white peony and 134 mg passion flower) and GABA-A receptors at concentrations of 0.01, 0.03, 0.1, 0.3, 1, 3, and 10 μg/ml.

FIG. 10 is a graph illustrating the percent of specific binding between Formula 3 (650 mg white peony and 200 mg passion flower) and GABA-A receptors at concentrations of 0.1, 0.3, 1, 3, and 10 μg/ml.

FIG. 11 is a graph illustrating the percent of specific binding between Formula 4 (400 mg white peony and 200 mg passion flower) and GABA-A receptors at concentrations of 0.1, 0.3, 1, 3, and 10 μg/ml.

FIG. 12 is a graph illustrating the percent of specific binding between Formula 5 (525 mg white peony and 200 mg passion flower) and GABA-A receptors at concentrations of 0.1, 0.3, 1, 3, and 10 μg/ml.

FIG. 13 is a graph illustrating the percent of specific binding between Formula 6 (250 mg white peony, 500 mg wild jujube) and GABA-A receptors at concentrations of 0.01, 0.03, 0.1, 0.3, 1, 3, and 10 μg/ml.

FIG. 14 is a graph illustrating the percent of specific binding between Formula 7 (650 mg white peony, 200 mg wild jujube) and GABA-A receptors at concentrations of 0.1, 0.3, 1, 3, and 10 μg/ml.

FIG. 15 is a graph illustrating the percent of specific binding between Formula 8 (700 mg wild jujube and 134 mg passion flower) and GABA-A receptors at concentrations of 0.01, 0.03, 0.1, 0.3, 1, 3, and 10 μg/ml.

FIG. 16 is a graph illustrating the percent of specific binding between Formula 9 (500 mg wild jujube and 134 mg passion flower) and GABA-A receptors at concentrations of 0.01, 0.03, 0.1, 0.3, 1, 3, and 10 μg/ml.

FIG. 17 is a graph illustrating the percent of specific binding between Formula 10 (375 mg white peony, 500 mg wild jujube, and 134 mg passion flower) and GABA-A receptors at concentrations of 0.01, 0.03, 0.1, 0.3, 1, 3, and 10 μg/ml.

FIG. 18 is a graph illustrating the percent of specific binding between Formula 11 (650 mg white peony, 200 mg wild jujube, and 200 mg passion flower) and GABA-A receptors at concentrations of 0.01, 0.03, 0.1, 0.3, 1, 3, and 10 μg/ml.

FIG. 19 is a graph illustrating the percent of specific binding between a control of White peony root and GABA-A receptors at concentrations of 0.01, 0.03, 0.1, 0.3, 1, 3, and 10 μg/ml.

FIG. 20 is a graph illustrating the percent of specific binding between an extract of a white peony root which does not contain paeoniflorin and GABA-A receptors at concentrations of 0.01, 0.03, 0.1, 0.3, 1, 3, and 10 μg/ml.

FIG. 21 is a graph illustrating the percent of specific binding between an extract of a white peony root which does not contain paeoniflorin and GABA-A receptors at concentrations of 0.01, 0.03, 0.1, 0.3, 1, 3, and 10 μg/ml.

FIG. 22 is a graph illustrating the percent of specific binding between an extract of white peony root which does not contain paeoniflorin and GABA-A receptors at concentrations of 0.01, 0.03, 0.1, 0.3, 1, 3, and 10 μg/ml.

FIG. 23 is a graph illustrating the percent of specific binding of a fraction of white peony extract root comprised of >90% paeoniflorin and GABA-A receptors at concentrations of 0.01, 0.03, 0.1, 0.3, 1, 3, and 10 μg/ml.

FIG. 24 is a graph illustrating the percent of specific binding of a fraction of white peony root comprised of >90% paeoniflorin and GABA-A receptors at concentrations of 0.01, 0.03, 0.1, 0.3, 1, 3, and 10 μg/ml.

### DETAILED DESCRIPTION

It is to be understood that this invention is not limited to the particular methodology or protocols described herein. Further, unless defined otherwise, all technical and scientific terms used herein have the same meaning as commonly understood to one of ordinary skill in the art to which this invention belongs. It is also to be understood that the terminology used herein is for the purpose of describing particular embodiments only, and is not intended to limit the scope of the present invention, which will be limited only by the claims.

The present invention relates to novel methods and compositions for the treatment of insomnia or sleeplessness. As used herein, unless otherwise specified, “treating insomnia” or “treating sleeplessness” includes, but is not limited to, preventing or reducing the disturbances in falling asleep, increasing the ability to stay asleep, reducing awakenings during sleep, increasing the duration and quality of sleep, and preventing or reducing abnormal sleep behaviors.

The methods and compositions of the present invention are based on the surprising discovery that unique combinations of jujube extract, white peony extract, radix polygala extract, and passion flower extract, which are discussed more fully below, work synergistically to shorten the time needed to fall asleep and improve the quality of sleep by reducing the number of awakenings during sleep and by increasing the duration of sleep. The present invention has the advantage of comprising natural, plant-based ingredients, which are non-addictive and which do not cause withdrawal when use is discontinued.

One extract used in compositions of the present invention, jujube, comes from leaves of the jujube tree, which originated in China but is now common to the southeast United States as well as California. Another jujube extract that may be used in the present invention comes from the jujube seeds. Jujube is a member of the Jujuba botanical group. Other members of this group include Semen zizphi spinosulae, Zizphi spinosulae Hu, Zizyphus jujube Mill var. spinosa Hu; Zizyphus jujube Mill var. spinosa Bunge; Zizyphus jujube spinosa (Bunge) Hu; Rhamnus jujube; Rhamnus zizyphus; Zizyphus mauritiana; Zizyphus spinosa; Zizyphus vulgaris var. sponosa; Zizyphus jujube; Zizyphus lotos; Zizyphus saiva; Zizyphus vulgaris; Zizyphus zizyphus. U.S. Patent Application Publication No. 2002/009506.

Jujube extract may contain sucrose, mucus, malic acid, tartaric acid, saponins, flavonoids, and other ingredients. Jujube extract is known to have the actions of recovering from fatigue, preventing excitation of nerves to allow mental stabilization, and relieving drug effects. U.S. Patent Application Publication No. 2004/0185118. See also U.S. Patent Application Publication No. 2002/0188025. Thus, jujube is used in compositions for improving brain function, increasing alertness, increasing memory, and reducing fatigue. U.S. Patent Application Publication No. 2002/0009506. It is also an organic source of cyclooxygenase-2 inhibitor and therefore, can be used to mediate inflammation or to treat an inflammation-associated disorder. U.S. Patent Application Publication No. 2002/0136784. According to the present invention, extracts of jujube also may be used to reduce the frequency of awakenings during sleep, induce sleep, shorten the time needed to fall asleep and increase the duration of sleep.

Paeoniflorin is another ingredient used in the compositions of the present invention. Paeoniflorin may be extracted, for example, from white peony root. Paeoniflorin is a unique glycoside present in peony root, that shortens the time needed to fall asleep, reduces the frequency of awakenings during sleep, and increases the duration of sleep. In one example, paeoniflorin may be extracted from any of the following: the root of Paeonia albiflora Pall. (P. Lactiflora Pall.), the root of P. Suffruticosa Andr. (P. moutan Sims), or the root of P. Delavayi Franch. An extract of a peony root may contain carbohydrates, proanthocyanidins, flavonoids, β-sitosterin, glycosides, benzene carboxylic acid, tannins, polysaccharides, and volatile oils. U.S. Patent Application Publication No. 2003/0232102. Both paeoniflorin and white peony extract have been shown to enhance mental function in animal studies. Ohta H, Ni J W, Matsumoto K, et al., “Paeony and its major constituent, paeoniflorin, improve radial maze performance impaired by scopolamine in rats.” Pharmacol Biochem. Behav. 1993. 45:719-23.

Although extracts of white peony root, which have sedation, pain relief, and anti-inflammation abilities, are known for use in arthritis treatments (see U.S. Patent Application Publication No. 2003/0232102 and U.S. Patent Application Publication No. 2003/0224073), and although white peony root is used in combination with other herbs in Traditional Chinese Medicine to treat insomnia resulting from anxiety, it is not known to use paeoniflorin, a specific compound present in white peony root, to bind GABA receptors and thereby shorten the time needed to fall asleep, reduce the number of awakenings during sleep, or increase the duration of sleep. Indeed, no particular component of white peony root has been identified as inducing and improving sleep or as binding GABA receptors.

In Traditional Chinese Medicine, white peony root is typically administered as a water decoction. A decoction is a concentrated form of tea. Typically, the dosage amount for a single day of a particular herb, such as white peony root, or of a combination of herbs, such as white peony root, jujube seed, radix polygala, and passionflower, are combined in a tea bag. The bag containing the herb or combination of herbs is boiled in water for approximately 30-60 minutes and the resulting decoction is consumed several times during the day. In this manner, the typical dosage of white peony root in Traditional Chinese Medicine is from approximately 6-15 g/day.

Radix polygala extract or Radix polygalae, also may be used in the compositions of the present invention based on its ability to shorten the time needed to fall asleep, reduce the number of awakenings during sleep, and increase the duration of sleep. Radix polygala is traditionally used in China and Korea as a sedative, anti-inflammatory agent, and antibacterial agent. It is also known for promoting mental stability. Additionally, Radix polygala has been shown to prevent N-methyl-D-aspartate induced neuronal cell damage and death in vitro over concentration ranges of 0.05 to 5 μg/ml. Lee H J, Ban J Y, Koh S B, Seong N S, Song K S, Bae K W, and Seong Y H. “Polygalae radix extract protects cultured rat granule cells against damage induced by NMDA,” Am. J. Chin. Med. 2004. 32(4):599-610.

Passion flower extract, which is known to have sleep promoting effects, also may be used in compositions of the present invention. Passion flower extract contains flavonoids, sterols, cholorogenic acid, volatile oil, and traces of alkaloids, including harmine, harman, harmol, harmaline, harmalol, and passaflorine. Passion flower extract is known to have anti-anxiety effects and is useful for reducing restlessness and nervousness. U.S. Patent Application Publication No. 2004/0185014. Indeed, when combined with Rhodiola crenulata, the sleep promoting effect of passion flower is enhanced. U.S. Patent Application Publication No. 2002/0127285.

Without being limited to any particular theory, it is believed that the white peony root extract containing paeoniflorin functions, at least in part, by binding to GABA receptors in the CNS. Binding of GABA receptors signals the CNS to relax. See Mohler, H., et al., 2001. “GABA-receptor subtypes: a new pharmacology.” Curr. Opin. in Pharmacology. 1:22-25. Indeed, many pharmaceutical treatments for insomnia, anxiety, sleeplessness, depression, and schizophrenia exert their effects at chemical synapses and many function by binding to transmitter-gated channels. For example, both barbiturates and tranquilizers, such as Valium® and Librium®, bind to GABA receptors, potentiating the inhibitory action of GABA by allowing lower concentrations of this neurotransmitter to open Cl⁻ channels, which thereby suppresses neuronal firing. It also is believed that jujube and radix polygala extracts, which may be used in compositions and methods of the present invention, also function, at least in part, by binding to GABA receptors in the CNS.

Significantly, of the extracts used in the compositions of the present invention, only passion flower extract is known to exert an effect on GABA receptors. Specifically, maltol and gamma-pyrone derivatives of passion flower extract are known to activate GABA receptors. Dhawan K, Kumar S, Sharma A. “Anti-anxiety studies on extracts of passiflora incarnate linneaus.” J. Ethnopharmacol 2001. 78:165-70. However, as demonstrated by the examples discussed below, white peony root extract, specifically, paeoniflorin, extracts of jujube seeds and/or leaves, and extracts of radix polygala bind to GABA receptors and exert relaxation and sleep inducing effects through such binding.

Compositions of the Present Invention

Therefore, in one embodiment, the present invention is a composition comprising approximately 10 mg-1000 mg of a paeoniflorin ingredient and an acceptable carrier. In other examples, the present invention may comprise approximately 10 mg-520 mg, 300 mg-1000 mg, 400 mg-850 mg, or 500 mg-650 mg of the paeoniflorin ingredient, and an acceptable carrier, wherein the paeoniflorin is effective for treating insomnia or sleeplessness by binding GABA receptors.

In another embodiment, the present invention is a composition comprising the following active ingredients in the following amounts:
10 mg-520 mg of a paeoniflorin ingredient and one or more of the following:
300 mg-1000 mg of jujube extract or any of its derivatives, or
100 mg-800 mg of passion flower extract or any of its derivatives,
   wherein the composition is effective for treating insomnia or sleeplessness.

In a further embodiment, the present invention is a composition comprising at least one of paeoniflorin, or jujube extract or any of its derivatives, wherein the composition binds strongly to GABA receptors.

In another embodiment, the present invention is a composition comprising the following active ingredients in the following amounts:
100 mg-800 mg of passion flower extract or any of its derivatives and one or more of the following:
10 mg-520 mg of paeoniflorin or any of its derivatives, or
300 mg-1000 mg of jujube extract or any of its derivatives,
   wherein the composition binds strongly to GABA receptors.

In a further embodiment, the present invention is a method of treating insomnia or sleeplessness comprising administering to a subject a composition comprising the following active ingredients in the following amounts:
10 mg-520 mg of paeoniflorin or any of its derivatives and one or more of the following:
300 mg-1000 mg of jujube extract or any of its derivatives,
300 mg-2000 mg of radix polygala extract or any of its derivatives, or
100 mg-800 mg of passion flower extract or any of its derivatives.

In another embodiment, the present invention is a method of treating insomnia or sleeplessness comprising administering to a subject a composition that binds strongly to GABA receptors, wherein the composition comprises the following active ingredients in the following amounts:
10 mg-520 mg of paeoniflorin or any of its derivatives and one or more of the following:
350 mg-1000 mg of jujube extract or any of its derivatives, or
100 mg-800 mg of passion flower extract or any of its derivatives.

The extracts used in the compositions of the present invention may be obtained from any commercially available source. For example, jujube extract is commercially available from Plum Flower Brand® Corp., white peony extract is commercially available from Organic Herb, Inc.®, Radix polygala extract is commercially available from Botanicum®, and passion flower extract is commercially available from Hammer Pharma®.

Alternatively, the extracts used in the compositions of the present invention may be obtained using any known extraction methods. For example, a jujube extract can be produced by extracting jujube leaves, fruits, bark, root, seeds etc. with an organic solvent. Some examples of organic solvents that might be used in producing the jujube extract to be used in the present invention include hexane, ethyl acetate, ethanol, and hydro-ethanol

In another example, solvent sequential fractionation may be used to obtain an extract of jujube, white peony, radix polygala, or passion flower. For example, using this technique, the leaves, fruit, bark, root, seeds, etc. of jujube can be sequentially extracted with hexane, ethyl acetate, ethanol, and hydro-ethanol. The extracts obtained after each step (fractions) of the sequence will contain chemical compounds in increasing order of polarity similar to the solvents used for extracting them. The fractions are dried to evaporate the solvents, resulting in a jujube extract. Those of skill in the art will appreciate that many other solvents can be used in practicing the solvent sequential fractionation extraction of any of the extracts used in practicing the present invention.

Total hydro-ethanolic extraction techniques might also be used to obtain an extract used in the compositions of the present invention. Generally, this is referred to as a lump-sum extraction of a material of interest, for example lump-sum extraction of a jujube leaf. The extract generated in this process will contain a broad variety of phytochemicals present in the material to be extracted, including fat and water solubles. Following collection of the extract, the solvent will be evaporated, resulting in an extract used in the compositions of the present invention.

Total ethanol extraction may also be used in the present invention. This technique also uses plant material to obtain the extract of interest, but ethanol, rather than hydro-ethanol, is the solvent. This extraction technique generates an extract that may include fat soluble and/or lipophilic compounds in addition to water soluble compounds.

Another example of an extraction technique that might be used to obtain one of the extracts used in the compositions of the present invention is supercritical fluid carbon dioxide extraction (SFE). In this extraction procedure the plant material containing the extract of interest is not exposed to any organic solvents. Rather, the extraction solvent is carbon dioxide, with or without a modifier, in supercritical conditions (>31.3° C. and >73.8 bar). Those of skill in the art will appreciate that temperature and pressure conditions can be varied to obtain the best yield of extract. This technique generates an extract of fat soluble and/or lipophilic compounds, similar to the total hexane and ethyl acetate extraction technique described above.

Those of skill in the art will appreciate that there are many other extraction processes, both known in the art and described in various patents and publications that can be used to obtain the extracts to be used in practicing the present invention. For example, the extraction procedures described in the following references, which are incorporated herein by reference, could be used in practicing the present invention: Wang et al., “Extraction and Chromatography-Mass Spectromic Analysis of the Active Principles from Selected Chinese Herbs and Other Medicinal Plants.” 2003. Am. J. Chin. Med. 31(6):927-44; Murga et al., “Extraction of natural complex phenols and tannins from grape seeds by using supercritical mixtures of carbon dioxide and alcohol.” J. Agric Food Chem. 2000 August:48(8):3408-12; Hong et al., “Microwave-assisted extraction of phenolic compounds from grape seed.” Nat Prod Lett. 2001;15(3):197-204; Ashraf-Khorassani et al., “Sequential fractionation of grape seeds into oils, polyphenols, and procyanidins via a single system employing CO₂-based fluids.” J. Agric Food Chem., 2004 May 5;52(9):2440-4.

The compositions of the present invention additionally may contain various known and conventional adjuvants so long as they do not detrimentally affect the sleep promoting effects provided by the composition. For example, a composition of the present invention can further include one or more additives or other optional ingredients well known in the art, which can include but are not limited to fillers (e.g., solid, semi-solid, liquid, etc.); carriers; diluents; thickening agents; gelling agents; vitamins, retinoids, and retinols (e.g., vitamin B₃, vitamin A, etc.); pigments; fragrances; anti-oxidants and radical scavengers; organic hydroxy acids; preservatives; antimicrobial agents; amino acids such as proline, pyrrolidone carboxylic acid, its derivatives and salts, saccharide isomerate, panthenol, buffers together with a base such as triethanolamine or sodium hydroxide; waxes, such as beeswax, ozokerite wax, paraffin wax; plant extracts, such as Aloe Vera, cornflower, witch hazel, elderflower, or cucumber and combinations thereof. Other suitable additives and/or adjuncts are described in U.S. Pat. No. 6,184,247, the entire contents of which are incorporated herein by reference.

The compositions of the present invention can include additional inactive ingredients, including, but not limited to surfactants, co-solvents, and excipients. Particular surfactants can be used based on the on the overall composition of the formulation and the intended delivery of the formulation. Useful surfactants include polyethoxylated (“PEG”) fatty acids, PEG-fatty acid diesters, PEG-fatty acid mono- and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, polysaccharide esters, polyethylene glycol alkyl phenols, polyoxyethylene-polyoxypropylene block copolymers, sorbitan fatty acid esters, lower alcohol fatty acid esters, ionic surfactants, and mixtures thereof.

The compositions of the present invention also can include co-solvents such as alcohols and polyols, polyethylene glycols ethers, amides, esters, other suitable co-solvents, and mixtures thereof. The compositions also can include excipients or additives such as sweeteners, flavorants, colorants, antioxidants, preservatives, chelating agents, viscomodulators, tonicifiers, odorants, opacifiers, suspending agents, binders, and mixtures thereof.

Methods of Administration

Compositions of the present invention may be formulated in an acceptable carrier and may be prepared, packaged, and labeled for treatment, prevention, or management of insomnia, sleeplessness, or symptoms thereof.

If a composition of the present invention is water-soluble, then it may be formulated in an appropriate buffer, for example, phosphate buffered saline or other physiologically compatible solutions. Alternatively, if a composition of the invention has poor solubility in aqueous solvents, then it may be formulated with a non-ionic surfactant such as Tween® or polyethylene glycol. Thus, the compositions of the present invention and their acceptable carriers may be formulated for oral administration in the form of a pill, tablet, powder, bar, food, beverage, lozenge, etc. The compositions of the present invention may also be parenterally administered or administered by inhalation or insufflation (either through the mouth or nose).

Compositions of the present invention may be orally administered in a liquid form such as a solution, syrup, beverage, or suspension. Additionally, compositions of the present invention may be presented as a dried or powdered product for reconstitution with water or other suitable vehicle before use. Liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid).

When administered in the form of a beverage, compositions of the present invention may be water-based, milk-based, tea-based, fruit juice-based, or some combination thereof.

Compositions of the present invention may also be orally administered in the form of a solid prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The solids may be coated by methods well-known in the art. In a preferred embodiment, the pharmaceutical composition may take the form of a capsule or powder to be dissolved in a liquid for oral consumption. Preparations for oral administration may be suitably formulated to give controlled release of the active compound.

Compositions of the present invention that are orally administered can further comprise thickeners, including xanthum gum, carbosymethyl-cellulose, carboxyethylcellulose, hydroxypropylcellulose, methylcellulose, microcrystalline cellulose, starches, dextrins, fermented whey, tofu, maltodextrins, polyols, including sugar alcohols (e.g., sorbitol and mannitol), carbohydrates (e.g. lactose), propylene hlycol alginate, gellan gum, guar, pectin, tragacanth gum, gum acacia, locust bean gum, gum arabic, gelatin, as well as mixtures of these thickeners. These thickeners are typically included in the formulations of the present invention at levels up to about 0.1%, depending on the particular thickener involved and the viscosity effects desired.

Orally administered compositions of the present invention can, and typically will, contain an effective amount of one or more sweeteners, including carbohydrate sweeteners and natural and/or artificial no/low calorie sweeteners. The amount of the sweetener used in the formulations of the present invention will vary, but typically depends on the type of sweetener used and the sweetness intensity desired.

In addition to the formulations described previously, the compounds may also be formulated as a sustained and/or timed release formulation. The compositions must be maintained above some minimum therapeutic dose to be effective. Common timed and/or controlled release delivery systems include, but are not be restricted to, starches, osmotic pumps, or gelatin micro capsules.

The compositions may, if desired, be presented in a pack or dispenser device which may comprise one or more unit dosage forms comprising a composition of the present invention. The pack may for example comprise metal or plastic foil, such as a blister pack. The pack or dispenser device may be accompanied by instructions for administration.

Other useful dosage forms can be prepared by methods and techniques that will be well understood by those of skill in the art and may include the use of additional ingredients in producing tablets, capsules, or liquid dosage forms. The dose, and dose frequency, will vary according to the age, body weight, condition and response of the individual consumer or patient, and the particular composition of the present invention that is used.

It is intended that the foregoing detailed description be regarded as illustrative rather than limiting. The present invention is further illustrated by the following experimental investigations and examples, which should not be construed as limiting. The contents of all references, patents and published applications cited throughout this patent are hereby incorporated by reference herein.

### EXAMPLES

### Example 1

### Ability of Valerian Extract to Strongly Bind GABA Receptors

Valerian, a medicinal herb that produces anxiolytic and sedative effects, is known to bind to GABA receptors and thereby potentiate the sedative effects of anesthetics and other medications that act on GABA receptors. See Yuan, Chun-Su, et al., 2004. “The Gamma-Aminobutryic Acidergic Effects of Valerian and Valerenic Acid on Rat Brainstem Neuronal Activity.” Anesth. Analg. 98:353-8.

One of ordinary skill in the art will appreciate that there are numerous methods for measuring the ability of a substance to bind GABA receptors. One example of such a method is set forth below and described in Yuan, Chun-Su, et al., 2004. “The Gamma-Aminobutryic Acidergic Effects of Valerian and Valerenic Acid on Rat Brainstem Neuronal Activity.” Anesth. Analg. 98:353-8, the entire contents of which are incorporated by reference herein. Yuan et al., confirm that the anxiolytic and sedative effects of valerian are due to valerian binding to GABA receptors.

In particular, at page 354, Yuan et al., explain that Sprague-Dawley neonatal rats 1 to 3 days old were deeply anesthetized with halothane. Next, a craniotomy was performed, and the forebrain was ablated by transaction at the caudal border of the pons. The caudal brainstem and cervical spinal cord were isolated by dissection in modified Krebs solution that contained (mM) NaCl 128.0, KCl 3.0, NaH₂PO₄ 0.5, CaCl₂ 1.5, MgSO₄ 1.0, NaHCO₃ 21, mannitol 1.0, glucose 30.0, and HEPES 10.0. The stomach connected to the esophagus, with the vagus nerves linking it to the brainstem, was kept, and all the other internal organs were removed. The preparation was then pinned with the dorsal surface upon a layer Sylgard resin (Dow Corning) in a recording chamber. The preparation was superfused with Krebs solution at 23° C.±1° C. The bathing solution was aerated continuously with a mixture of 95% oxygen and 5% CO₂ and adjusted to pH 7.35-7.45.

Single tonic unitary discharges were recorded extracellularly in the NTS by glass microelectrodes filled with 3 M NaCl, with an impedance of 10-20 Ω (unitary discharge recordings). One to five neurons were recorded from each preparation. A collision test was applied by stimulating the recorded unit and the subdiaphragmatic vagal nerve to identify orthodromic inputs, to ensure that only second- or higher-order NTS neurons in the afferent system were used in the experiment. For histological identification purposes, glass microelectrodes were filled with 2% pontamine sky blue in 0.5 M sodium acetate solution. After each unitary recording, current was applied at 5 μA in cycles of 5 s on/10 s off for approximately 5 min, with the negative lead connected to the microelectrode.

To independently evaluate the brainstem effects of GABA on NTS neurons, a partition was made at the thoracic level of the preparation. An agar seal formed a recording bath chamber of the brainstem compartment. Test substances, valerian extract in particular, were applied only to the brainstem compartment, and their effects on the NTS neuronal activity were evaluated. After each observation, the test substance was washed out from the compartment. The NTS neuronal responses observed during pretreatment (control) were compared with posttreatment (washout) to confirm that brainstem neuronal activity returned to the control level after washout.

In each experiment, the NTS unitary discharges were amplified with high-gain alternating current-coupled amplifiers (Axoprobe-1A; Axon Instruments, Burlingame, Calif.), displayed on a Hitachi digital storeage oscilloscope (Model VC-6525; Hitachi Denshi, Ltd., Japan), and recorded on a Vetter PCM tape recorder (Model 200; AR Vetter Co., Rebersburg, Pa.).

Valerian extract was obtained from Lichtwe Pharma AG (Berlin, Germany). The extract was standardized to 0.3% valerenic acids (which contained valerenic acid and acetyl and hydroxyvaleric acids) by the manufacturer. Valerenic acid (>98%) was obtained from ChromaDex, Inc. (Santa Ana, Calif.).

The data from the NTS unitary activity were analyzed on the basis of action potential discharge rate and test substance concentration-related effects. The number of action potentials in a given duration was measured under pretreatment, treatment, and posttreatment conditions (usually 50 s in each trial). The control data (pretrial) were normalized to 100% and the NTS neuronal activities during and after treatments were expressed in terms of the percentage of control activity. Application of 3 mg/ml of valerian extract induced an inhibitory effect of 29.6%±5.1%.

This experiment also measured the IC₅₀ value of valerian extract, which is the concentration of valerian extract required to displace 50% of a radio labeled ligand ([3H]-GABA) at 5.0 nM. The IC₅₀ value of valerian extract as measured in this experiment was 240±18.7 μg/ml.

One of ordinary skill in the art will appreciate that the methods of Yuan et al., may be used to test the ability of other extracts such as wild jujube, white peony, radix polygala, and/or passion flower, or any derivative thereof, to bind GABA receptors.

### Example 2

### Extraction of White Peony Root

Native white peony root, without pre-treatment or excipients, was ground into a powder and extracted thoroughly by refluxing the white peony root powder with methanol containing 5% water at approximately 80-84° C. for 3 hours.

The extracted solvent was concentrated using a rotary evaporator. The extraction residue was dissolved in a small amount of methanol to prepare an approximately 5 mg/ml concentration of white peony root extract. This solution was used for the Thin-Layer Chromatography analysis described in Example 3.

### Example 3

### Thin-Layer Chromatography Analysis of Extracts of White Peony Root

Thin-layer chromatography (TLC) plates having specifications of 20×20 cm and 1000 micron, coated with a preparative layer of silica with a UV rating of 254 (catalogue # 08002) were obtained from Analtech, Inc. (Newark, Del.). The TLC plates were coated with a mobile phase comprising methanol:chloroform:ethylacetate (v:v:v) in the ratio of 1:2:1.

The TLC plates were coated with the solution described above in Example 2 and the plates were allowed to dry. Different bands from the TLC plates were visualized by UV at 254 nm and collected by scraping the TLC plates. Separation was repeated by TLC until pure fractions (>92% paeoniflorin) were obtained. Individual fractions were subsequently analyzed by HPLC.

### Example 4

### Ability of Extracts from White Peony Root to Strongly Bind GABA Receptors

GABA receptor agonist binding assays were used to test the bioactivity of six different extracts of white peony root. These GABA-A Agonist assays were conducted by NovaScreen Biosciences Corporation (Hanover, Md.) and are described in Falch E., et al., 1986. “Comparative stereostructure-activity studies on GABA-A and GABA-B receptor sites and GABA uptake using rat brain membrane preparations.” J. Neurochem. 47(3): 898-903 and Enna S. J., et al., 1977. “Stereospecificity and structure-activity requirements of GABA receptor binding in rat brain.” Brain Res. 124(1): 185-190, which are incorporated by reference herein in their entirety.

In particular, the GABA-A Agonist binding assays use bovine cerebellar membranes as the source of GABA-A receptors. GABA-A, radio labeled with tritium (³H), is used as both the reference compound and positive control. Unlabeled GABA was used to establish a standard curve with a high concentration of 10 μM.

The bovine cerebellar membranes are exposed to both [³H]-GABA and extracts of the desired test substance. In this instance, the bovine cerebellar membranes were exposed to: six extracts of white peony root at concentrations of 0.01, 0.03, 0.1, 0.3, 1, 3, and 10 μg/ml. The GABA-A Agonist assay measures how much radio labeled GABA-A binds to the GABA-A receptors present in the bovine cerebellar membranes.

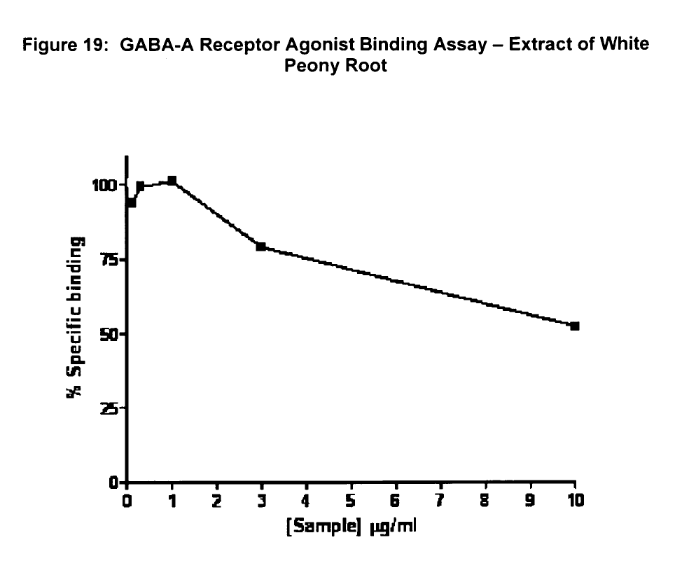

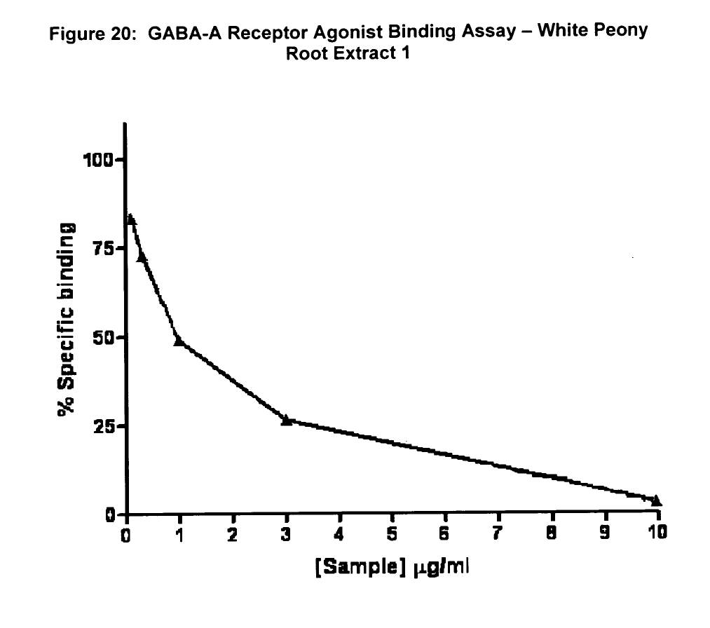

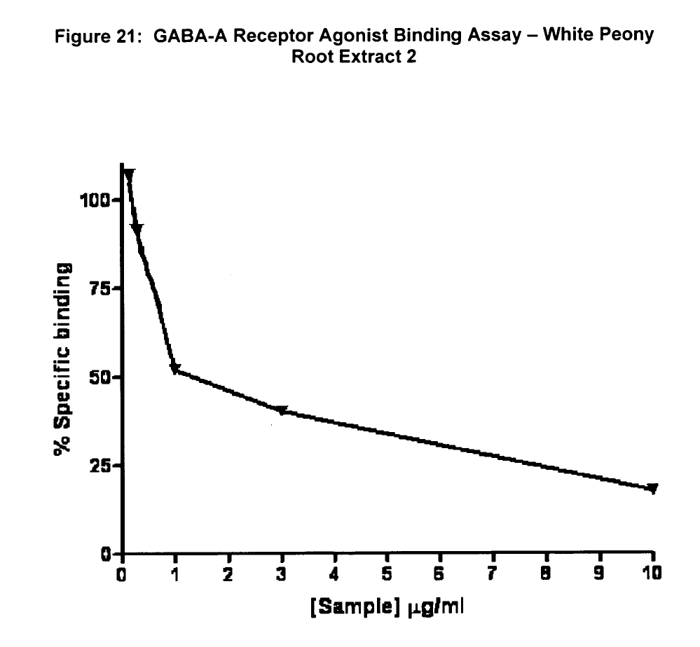

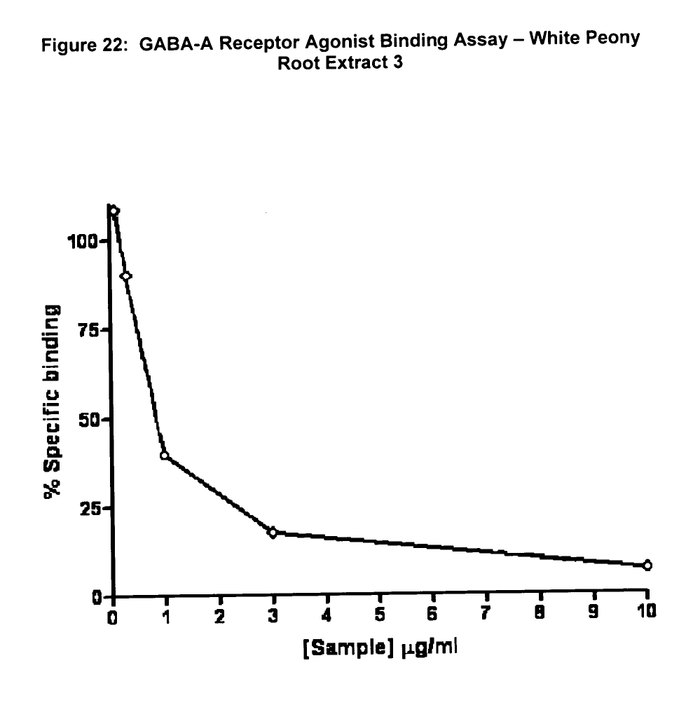

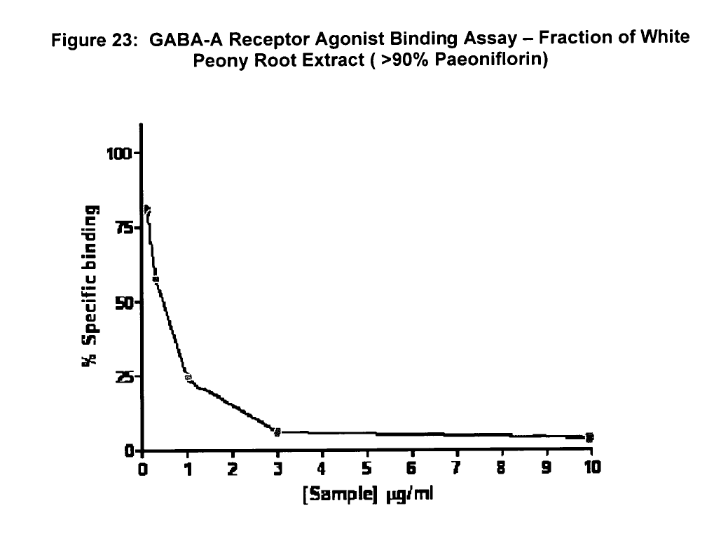

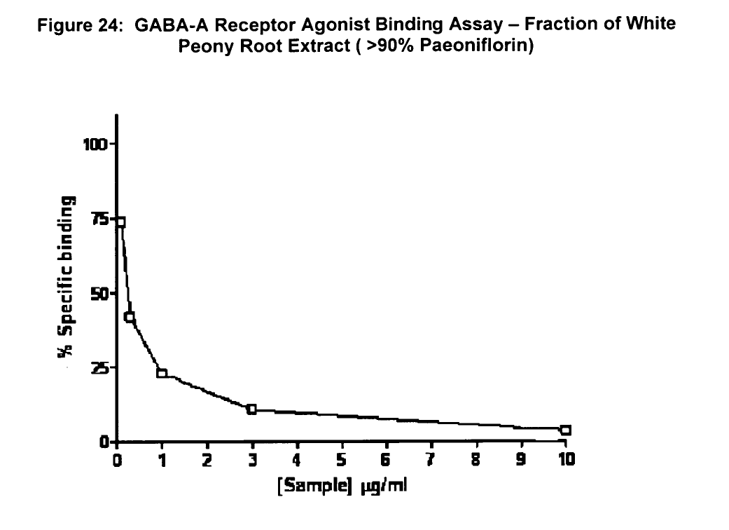

The results of the GABA-A Agonist binding assays are shown in FIGS. 19-24 and are expressed below in Table I as IC₅₀ in μg/ml. These results indicate the concentration of the sample tested that was required to displace 50% of the radio labeled ligand ([3H]-GABA) at 5.0 nM. These results demonstrate that extracts of white peony root exert anxiolytic and sedative effects via binding to the GABA receptor.

TABLE 1
Results of GABA Receptor Agonist Binding Assay - Test
Substance = Fractionation Samples of White Peony Extract

| Sample Number: | IC₅₀ μg/ml |
|---|---|
| 0 (no paeoniflorin) | >10 |
| 1 (no paeoniflorin) | 0.907 |
| 2 (no paeoniflorin) | 1.290 |
| 3 (no paeoniflorin) | 1.120 |
| >90% paeoniflorin | 0.373 |
| >90% paeoniflorin | 0.265 |

The results of this analysis demonstrate that the fractions containing paeoniflorin had significant binding activity (IC₅₀<0.4 μg/ml). When converted to molarity, the IC₅₀ for paeoniflorin is approximately 625 nM, a value very close to the K_{D} of GABA (370 nM) for the assay.

### Example 5

### Ability of Jujube Extract, White Peony Extract, Radix Polygala Extract, Passion Flower Extract, and Valerian Extract to Strongly Bind GABA Receptors

GABA receptor agonist binding assays were used to test the bioactivity of white peony extract, jujube extract, radix polygala extract, passion flower extract, valerian extract, or derivatives thereof. These GABA-A Agonist assays were conducted by NovaScreen Biosciences Corporation (Hanover, Md.) and are described in Falch E., et al., 1986. “Comparative stereostructure-activity studies on GABA-A and GABA-B receptor sites and GABA uptake using rat brain membrane preparations.” J. Neurochem. 47(3): 898-903 and Enna S. J., et al., 1977. “Stereospecificity and structure-activity requirements of GABA receptor binding in rat brain.” Brain Res. 124(1): 185-190, which are incorporated by reference herein in their entirety.

In particular, the GABA-A Agonist binding assays use bovine cerebellar membranes as the source of GABA-A receptors. GABA-A, radio labeled with tritium (³H), is used as both the reference compound and positive control. The bovine cerebellar membranes are exposed to both [³H]-GABA and extracts of the desired test substance. In this instance, the bovine cerebellar membranes were exposed to: wild jujube or any of its derivatives; white peony or any of its derivatives; radix polygala or any of its derivatives; or passionflower or any of its derivatives; or valerian extract or any of its derivatives at concentrations of 0.01, 0.03, 0.1, 0.3, 1, 3, and 10 μg/ml. The GABA-A Agonist assay measures how much radio labeled GABA-A binds to the GABA-A receptors present in the bovine cerebellar membranes.

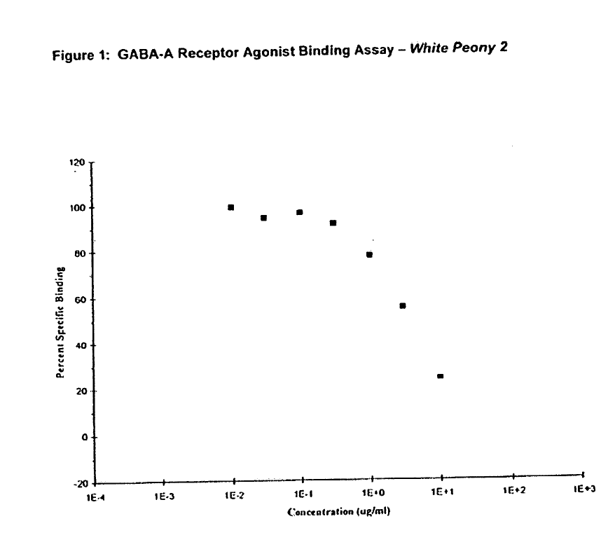

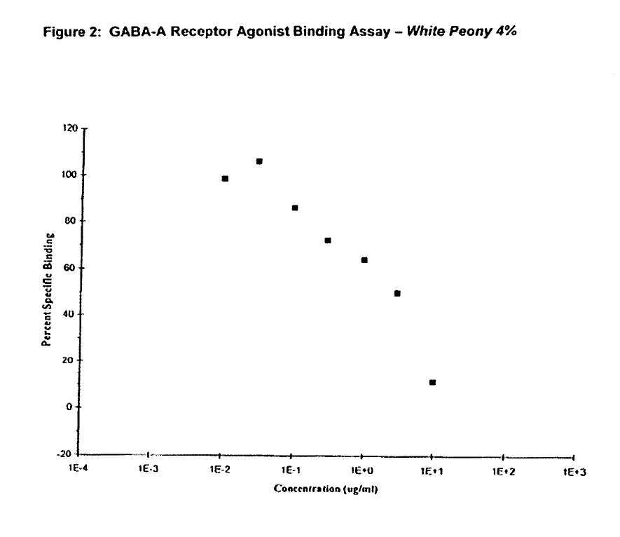

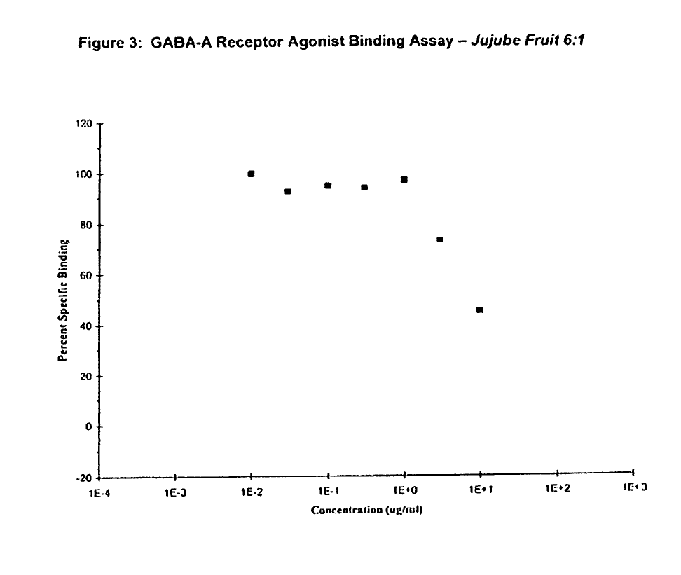

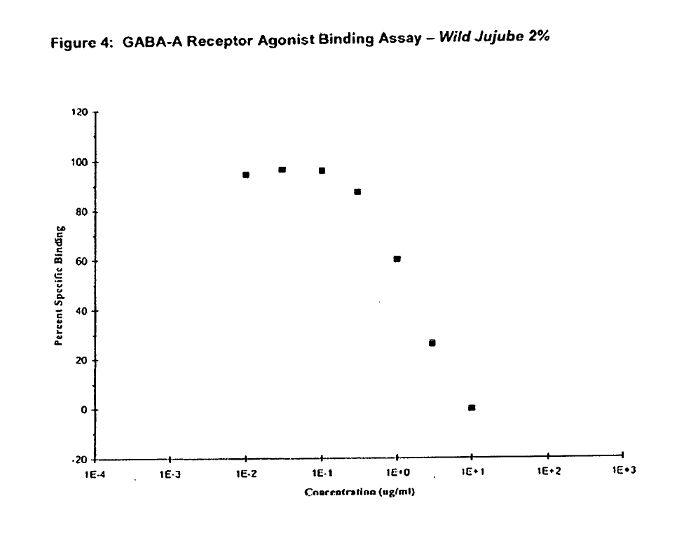

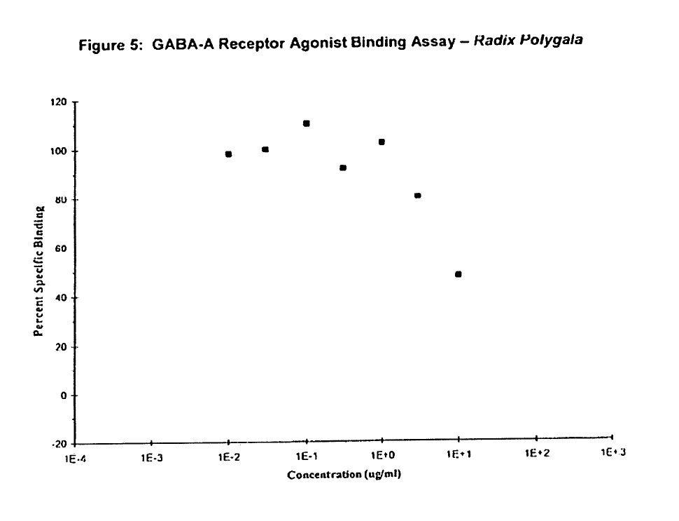

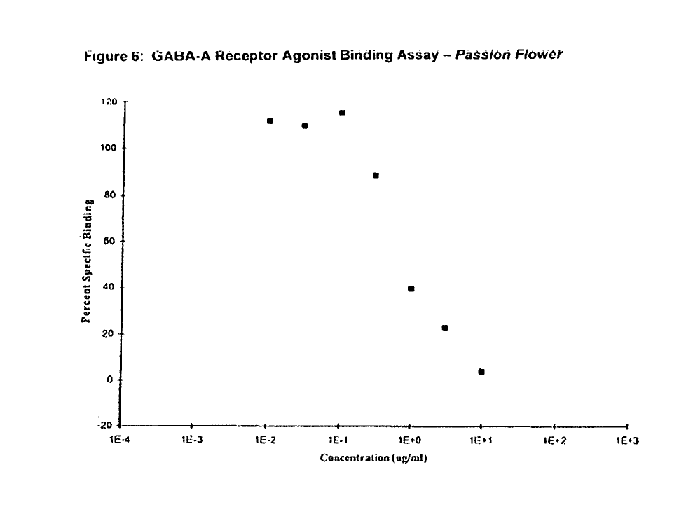

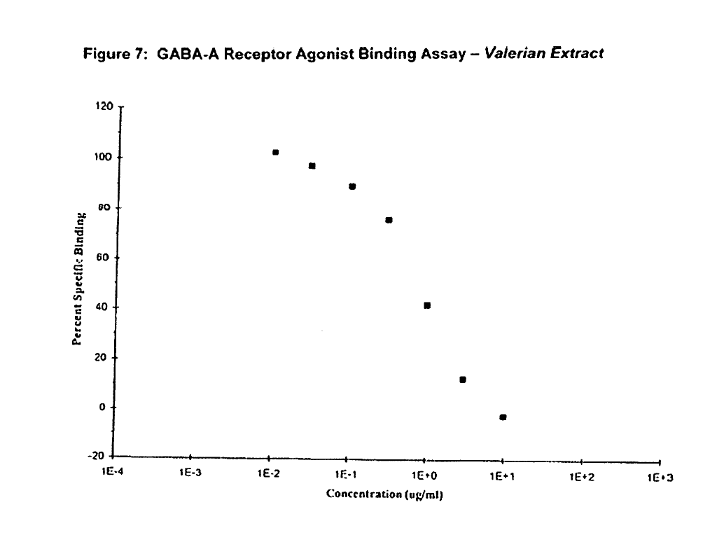

The results of the GABA-A Agonist binding assays are shown in FIGS. 1-7 and are expressed below in Table II as IC₅₀ in μg/ml. These results indicate the concentration of the sample tested that was required to displace 50% of the radio labeled ligand ([3H]-GABA) at 5.0 nM. These results demonstrate that extracts of white peony, wild jujube, and passion flower all have IC₅₀ values similar to valerian extract, which as discussed above, is known to exert anxiolytic and sedative effects via binding to the GABA receptor.

TABLE II
Results of GABA Receptor Agonist Binding Assays

| Sample Description | IC₅₀ in μg/ml |
|---|---|
| White Peony 2% | 3.45 |
| White Peony 4% | 1.46 |
| Jujube Fruit 6:1 | 8.24 |
| Wild Jujube Extract 2% | 1.35 |
| Radix Polygala | 9.31 |
| Passion Flower | 1.07 |
| Valerian Extract | 0.75 |

Further results of the GABA-A Antagonist Binding Assays, including the % inhibition (%I) and % specific binding (% SB) for each extract, are also shown in FIGS. 1-7 and are reported below in Table III.

TABLE III
Inhibition and Specific Binding Results of GABA Receptor
Agonist Binding Assays

| | 0.01 μg/ml | | 0.03 μg/ml | | 0.1 μg/ml | | 0.3 μg/ml | | 1 μg/ml | | 3 μg/ml | | 10 μg/ml | |
|---|---|---|---|---|---|---|---|---|---|---|---|---|---|---|
| | (1.0E−2) | | (3.0E−2) | | (1.0E−1) | | (3.0E−1) | | (1.0E−0) | | (3.0E−0) | | (1.0E−1) | |
| Sample | % I | % SB | % I | % SB | % I | % SB | % I | % SB | % I | % SB | % I | % SB | % I | % SB |
| White | .73 | 99.27 | 5.35 | 94.65 | 3.25 | 96.75 | 8.06 | 91.94 | 22.34 | 77.66 | 44.57 | 55.43 | 75.71 | 24.29 |
| Peony | | | | | | | | | | | | | | |
| 2% | | | | | | | | | | | | | | |
| White | 0.88 | 99.12 | −7.0 | 107.0 | 12.96 | 87.04 | 26.99 | 73.01 | 35.30 | 64.70 | 49.22 | 50.78 | 88.06 | 11.94 |
| Peony | | | | | | | | | | | | | | |
| 4% | | | | | | | | | | | | | | |
| Jujube | 0.0 | 100.0 | 7.13 | 92.87 | 4.87 | 95.13 | 5.88 | 94.12 | 2.82 | 97.18 | 26.86 | 73.14 | 54.76 | 45.24 |
| Fruit 6:1 | | | | | | | | | | | | | | |
| Wild | 5.22 | 94.78 | 3.30 | 96.70 | 3.73 | 96.27 | 12.62 | 87.38 | 39.72 | 60.28 | 74.03 | 25.97 | 100.25 | −0.25 |
| Jujube | | | | | | | | | | | | | | |
| 2% | | | | | | | | | | | | | | |
| Radix | 1.71 | 98.29 | 0.00 | 100.00 | −10.47 | 110.47 | 7.81 | 92.19 | −2.46 | 102.46 | 19.66 | 80.34 | 52.27 | 47.73 |
| Polygala | | | | | | | | | | | | | | |
| Passion | −12.09 | 112.09 | −9.99 | 109.99 | −15.57 | 115.57 | 11.11 | 88.89 | 60.37 | 39.63 | 77.04 | 22.96 | 96.25 | 3.75 |
| Flower | | | | | | | | | | | | | | |
| Valerian | −3.22 | 103.22 | 1.78 | 98.22 | 9.81 | 90.19 | 23.29 | 76.71 | 57.47 | 42.53 | 87.08 | 12.92 | 102.05 | −2.05 |

### Example 6

### Ability of Various Formulations of the Present Invention to Strongly Bind GABA Receptors

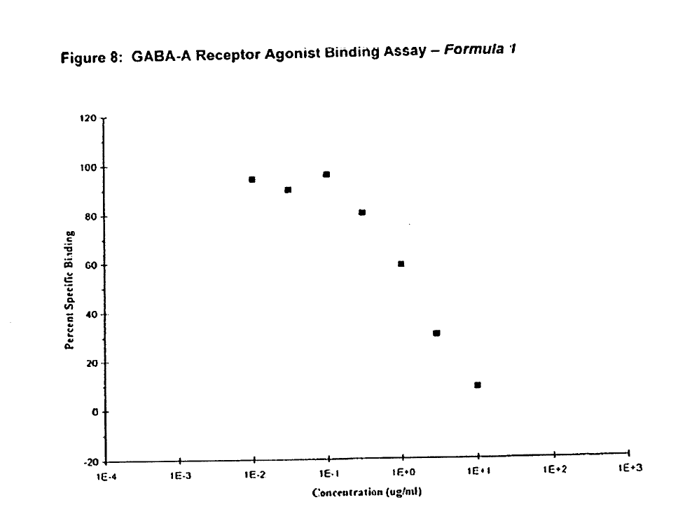

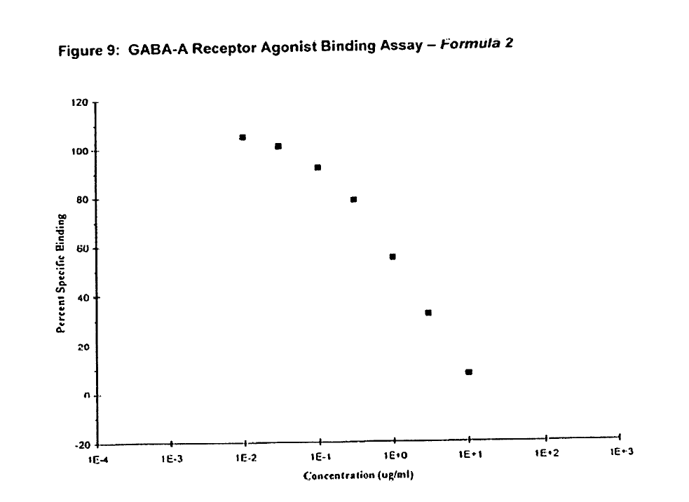

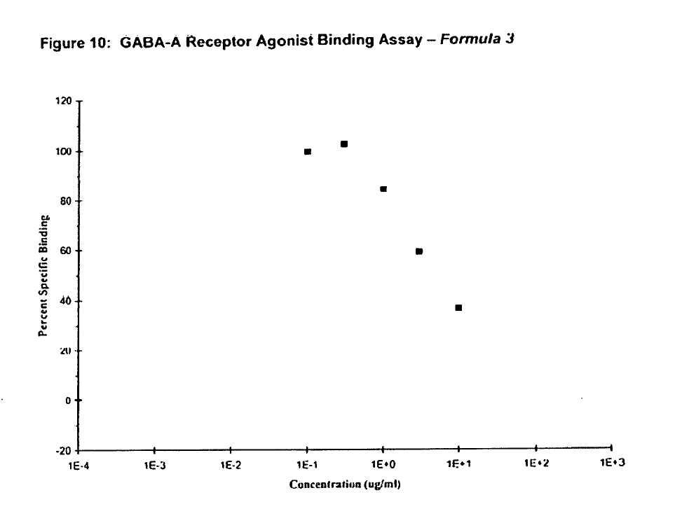

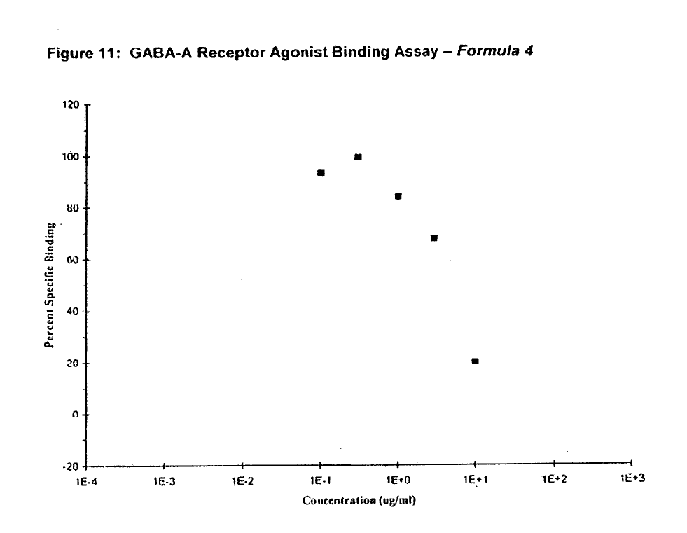

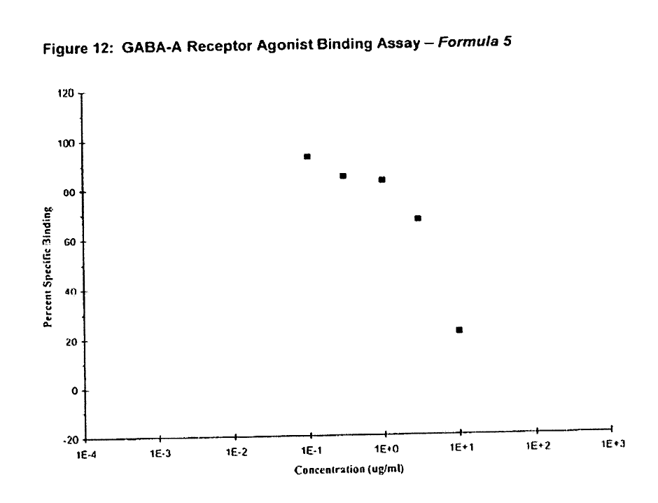

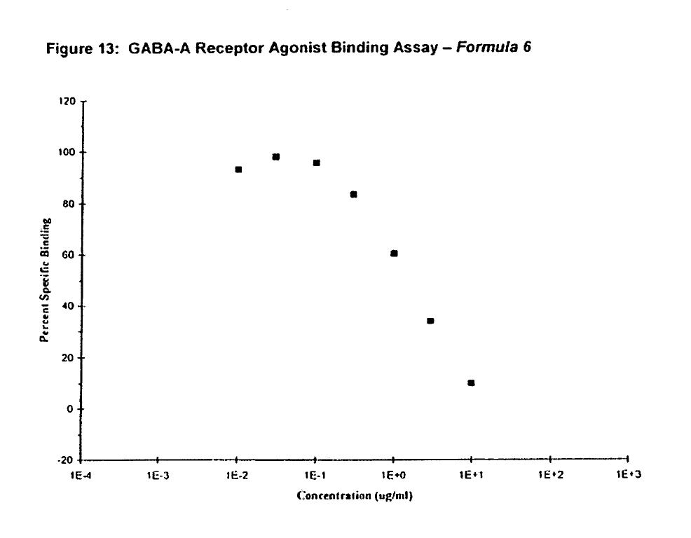

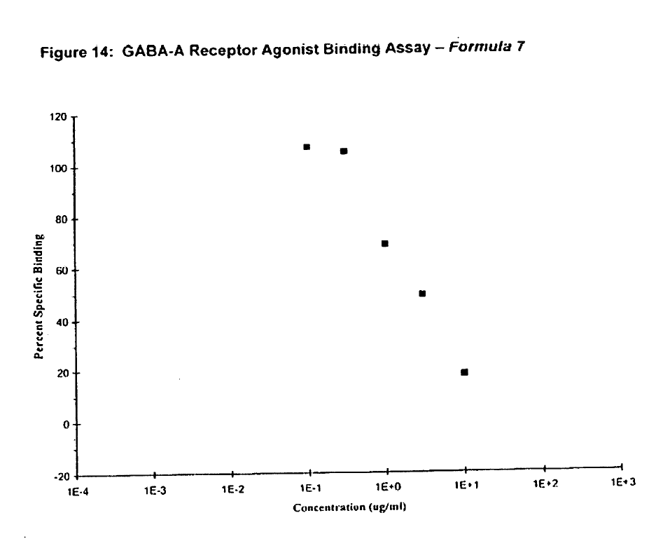

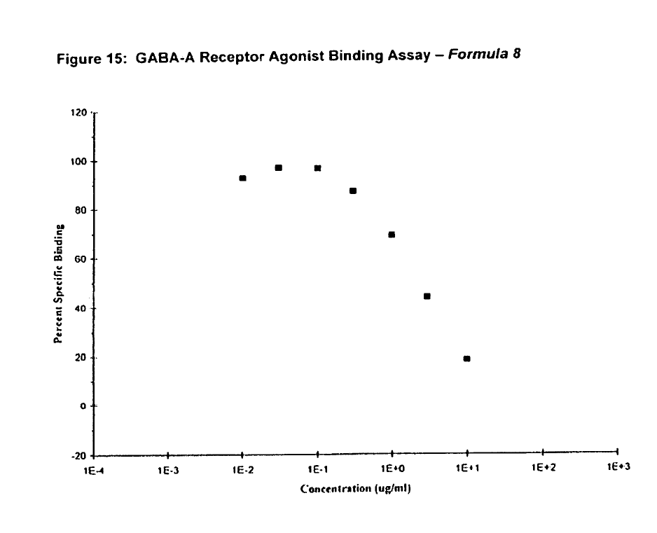

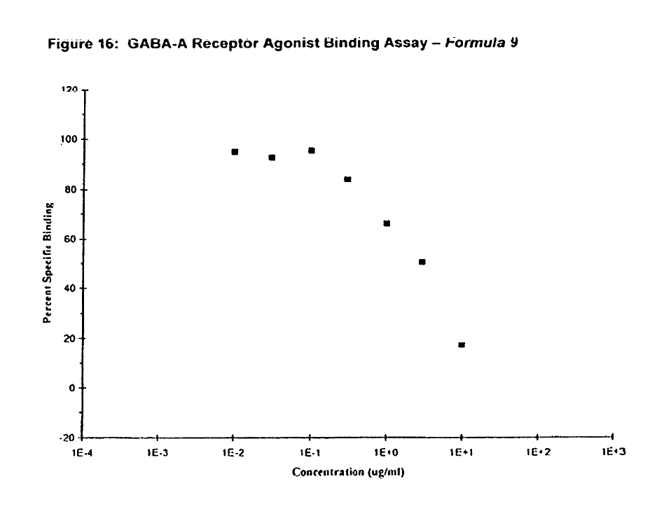

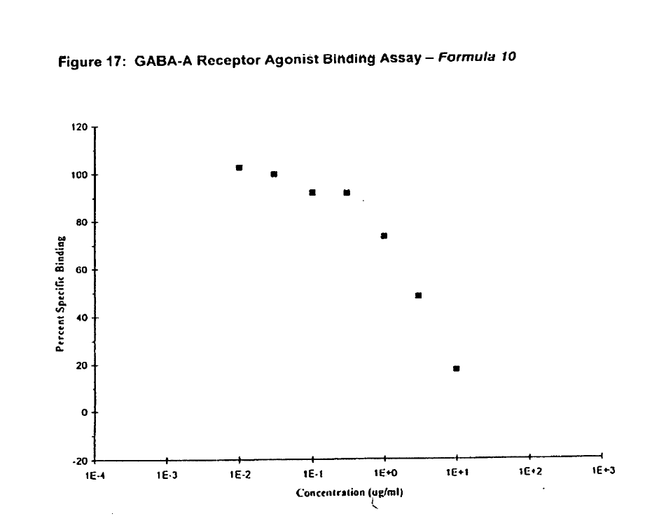

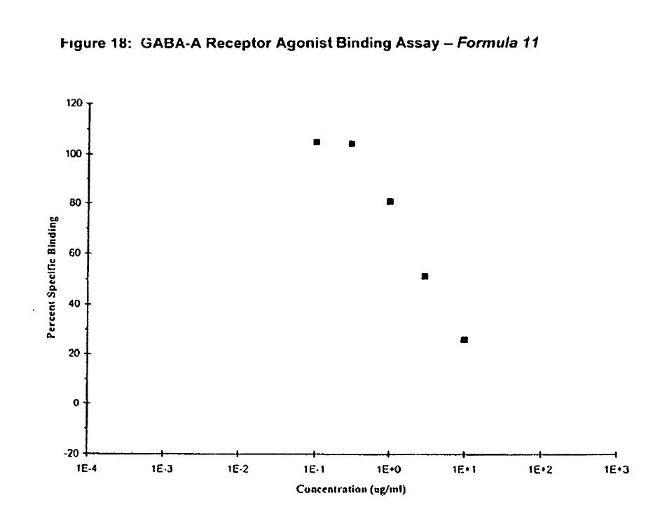

The GABA-A Agonist binding assays described above in Example 5 were repeated using the formulations described in Table IV. The results of these assays are shown in FIGS. 8-18 and are reported below in Table IV. These results, reported as IC₅₀ in μg/ml, indicate the concentration of the sample tested that was required to displace 50% of the radio labeled ligand ([3H]-GABA) at 5.0 nM. The results reported in Table IV demonstrate that Formulas 1, 2, 6, 8, 9, and 10 all have IC₅₀ values similar to valerian extract (0.75), which as discussed above, is known to exert anioxlytic and sedative effects via binding to the GABA receptor.

TABLE IV
Results of GABA Receptor Agonist Binding Assays

| Sample Description | IC₅₀ in μg/ml |
|---|---|
| Formula 1 (434 mg White Peony, 200 mg Passion Flower) | 0.74 |
| Formula 2 (375 mg White Peony, 134 mg Passion Flower) | 1.19 |
| Formula 3 (650 mg White Peony, 200 mg Passion Flower) | 5.3 |
| Formula 4 (400 mg White Peony, 200 mg Passion Flower) | 4.04 |
| Formula 5 (525 mg White Peony, 200 mg Passion Flower) | 3.89 |
| Formula 6 (250 mg White Peony, 500 mg Wild Jujube) | 1.84 |
| Formula 7 (650 mg White Peony, 200 mg Jujube) | 2.41 |
| Formula 8 (700 mg Jujube, 134 mg Passion Flower) | 1.67 |
| Formula 9 (500 mg Jujube, 134 mg Passion Flower) | 0.89 |
| Formula 10 (375 mg White Peony, 500 mg Jujube, 134 mg | 1.92 |
| Passion Flower) | |
| Formula 11 (650 mg White Peony, 200 mg Jujube, 200 mg | 3.61 |
| Passion Flower) | |

Further results of the GABA-A Antagonist Binding Assays, including the % inhibition (% I) and % specific binding (% SB) for each extract, are also shown in FIGS. 8-18 and are reported below in Table V.

TABLE V
Inhibition and Specific Binding Results of GABA Receptor
Agonist Binding Assays

| | | | | | | |
|---|---|---|---|---|---|---|
| 1.0E−2 | 3.0E−2 | 1.0E−1 | 3.0E−1 | 1.0E−0 | 3.0E−0 | 1.0E−1 |

| Sample | % I | % SB | % I | % SB | % I | % SB | % I | % SB | % I | % SB | % I | % SB | % I | % SB |
|---|---|---|---|---|---|---|---|---|---|---|---|---|---|---|
| Formula 1 | 5.58 | 94.42 | 10.04 | 89.96 | 4.17 | 95.83 | 19.78 | 80.22 | 41.38 | 58.62 | 69.71 | 30.29 | 91.21 | 8.79 |
| Formula 2 | −5.15 | 105.15 | −1.15 | 101.15 | 7.64 | 92.36 | 21.24 | 78.76 | 44.92 | 55.08 | 67.75 | 32.25 | 92.50 | 7.50 |
| Formula 3 | n/a | n/a | n/a | n/a | 0.31 | 99.69 | −2.72 | 102.72 | 15.27 | 84.73 | 40.62 | 59.38 | 63.16 | 36.84 |
| Formula 4 | n/a | n/a | n/a | n/a | 6.44 | 93.56 | 0.56 | 99.44 | 15.49 | 84.51 | 31.90 | 68.10 | 80.01 | 19.99 |
| Formula 5 | n/a | n/a | n/a | n/a | 7.05 | 92.95 | 14.98 | 85.02 | 16.89 | 83.11 | 33.07 | 66.93 | 78.16 | 21.84 |
| Formula 6 | 6.45 | 93.55 | 1.64 | 98.36 | 3.79 | 96.21 | 16.07 | 83.93 | 39.28 | 60.72 | 65.62 | 34.38 | 89.55 | 10.45 |
| Formula 7 | n/a | n/a | n/a | n/a | −7.14 | 107.1 | −5.24 | 105.24 | 31.16 | 68.84 | 51.00 | 49.00 | 82.06 | 17.94 |
| Formula 8 | 7.02 | 92.98 | 2.83 | 97.17 | 3.02 | 96.98 | 12.25 | 87.75 | 30.45 | 69.55 | 55.72 | 44.28 | 81.48 | 18.52 |
| Formula 9 | 4.85 | 95.15 | 6.99 | 93.01 | 4.25 | 95.75 | 15.60 | 84.40 | 33.61 | 66.39 | 49.33 | 50.67 | 82.63 | 17.37 |
| Formula 10 | −2.69 | 102.69 | −0.07 | 100.07 | 7.76 | 92.24 | 7.93 | 92.07 | 26.46 | 73.54 | 51.74 | 48.26 | 82.60 | 17.40 |
| Formula 11 | n/a | n/a | n/a | n/a | −5.22 | 105.2 | −4.69 | 104.69 | 18.74 | 81.26 | 48.58 | 51.42 | 73.30 | 26.30 |

1. A composition for binding GABA receptors comprising paeoniflorin and an acceptable carrier.

2. The composition of claim 1, wherein the composition is effective for treating insomnia or sleeplessness.

3. The composition of claim 2, wherein the paeoniflorin comprises at least 5% of a white peony extract.

4. The composition of claim 2, further comprising one or more of: an extract of jujube leaves or any derivatives thereof; an extract of jujube seeds or any derivatives thereof; an extract of radix polygala or any derivatives thereof; and an extract of passion flower or any derivatives thereof.

5. The composition of claim 4, wherein the white peony extract containing the paeoniflorin is present in an amount ranging from approximately 10 mg-1000 mg; and wherein, if present, the extract of jujube leaves or any derivatives thereof is present in an amount ranging from approximately 300 mg-1000 mg; the extract of jujube seeds or any derivatives thereof is present in an amount ranging from approximately 300 mg-1000 mg; the extract of radix polygala or any derivatives thereof is present in an amount ranging from approximately 300 mg-2000 mg; and the extract of passion flower or any derivatives thereof is present in an amount ranging from approximately 100 mg-800 mg.

6. The composition of claim 4, wherein the paeoniflorin is present in an amount ranging from approximately 10 mg-520 mg.

7. The composition of claim 4, wherein the composition is in the form of a pill, tablet, powder, food, beverage, or lozenge.

8. The composition of claim 4, further comprising vitamins A, C, E, B₆, and B₁₂, or derivatives thereof.

9. A method of treating insomnia or sleeplessness in a mammal comprising administering the composition of claim 5 to the mammal, wherein the paeoniflorin in the composition binds GABA receptors.

10. The method of claim 9, wherein the composition is orally administered in the form of a pill, tablet, powder, food, beverage, or lozenge.

11. The method of claim 9, wherein the beverage is water-based, milk-based, tea-based, fruit juice-based, or some combination thereof.

12. The method of claim 9, wherein the paeoniflorin comprises at least 5% of a white peony root extract.
